(19) United States
(12) Patent Application Publication
(10) Pub. No.: US 2016/0367221 A1
(43) Pub. Date: Dec. 22, 2016

(54) ULTRASOUND DIAGNOSIS APPARATUS

(71) Applicant: Toshiba Medical Systems Corporation, Otawara-shi (JP)

(72) Inventors: Yu IGARASHI, Utsunomiya (JP); Kazuya AKAKI, Utsunomiya (JP); Shunsuke SATOH, Nasushiobara (JP); Go TANAKA, Otawara (JP); ltsuki KUGA, Nasushiobara (JP); Takayuki GUNJI, Otawara (JP); Masaki WATANABE, Takanezawa (JP)

(73) Assignee: Toshiba Medical Systems Corporation, Otawara-shi (JP)

(21) Appl. No.: 15/183,153

(22) Filed: Jun. 15, 2016

(30) Foreign Application Priority Data
Jun. 16, 2015 (JP) 2015-121539

(51) Int. Cl. A61B 8/08; A61B 8/00; A61B 8/06; A61B 8/14

(52) U.S. Cl. CPC A61B 8/5207; A61B 8/145; A61B 8/5253; A61B 8/54; A61B 8/5223; A61B 8/466; A61B 8/4461; A61B 8/4494; A61B 8/469; A61B 8/06; A61B 8/463; A61B 8/4245; A61B 8/488

(57) ABSTRACT

An ultrasound diagnosis apparatus generates first data, based on a result of transmission and reception that are executed when a probe is located at a first position of a subject. The apparatus generates second data, based on a result of transmission and reception that are executed when the probe is located at a second position. Under a first constraint on an orientation of a section, the apparatus extracts, from the first data, first sectional image containing a structural object inside the subject and taken along a direction in which the object extends. Under a second constraint on the orientation of a section, the apparatus extracts, from the second data, second sectional image containing the object and taken along a direction in which the object extends. The apparatus generates joined image data composed of at east a part of the first sectional image and the second sectional image joined together.

### CROSS-REFERENCE TO RELATED APPLICATIONS

This application is based upon and claims the benefit of priority from Japanese Patent Application No. 2015-121539, filed on Jun. 16, 2015; the entire contents of which are incorporated herein by reference. The entire contents of the prior Japanese Patent Application No. 2016-117270, filed on Jun. 13, 2016, are also incorporated herein by reference.

### FIELD

Embodiments described herein relate generally to an ultrasound diagnosis apparatus.

### BACKGROUND

An ultrasound diagnosis apparatus is an apparatus that acquires biological information by emitting, into a subject, ultrasound pulses generated by piezoelectric transducer elements provided in an ultrasound probe and then receiving reflected ultrasound waves through the piezoelectric transducer elements. The reflected ultrasound waves are generated by differences in acoustic impedance of tissue in the subject. Ultrasound diagnosis apparatuses enable substantially real-time display of image data with a simple operation of only bringing an ultrasound probe into contact with a body surface, and therefore have been used in a board range of applications such as shape diagnosis and functional diagnosis on various organs.

There has been a technique for, when a region of interest (structural object) inside a subject is located across a range wider than a scanning region of an ultrasound probe, combining ultrasound image data acquired at plurality of locations into one to generate image data that covers a wide range. In this case, for example, an ultrasound diagnosis apparatus acquires image data for a plurality of frames through manipulation by an operator such that an ultrasound probe is moved little by little along a body surface and combines the image data for these frames into one, thereby generating image data (panoramic image data) that covers a wide range.

### BRIEF DESCRIPTION OF THE DRAWINGS

FIG. 1 is a block diagram illustrating an exemplary configuration of an ultrasound diagnosis apparatus according to a first embodiment;

FIG. 2 is a flowchart for explaining processing in the ultrasound diagnosis apparatus according to the first embodiment;

FIG. 3 is a diagram for explaining determination of an initial section according to the first embodiment;

FIG. 4A and FIG. 4B are diagrams for explaining determination of the initial section according to the first embodiment;

FIG. 5 is a diagram for explaining processing in a transmission/reception control unit according to the embodiment;

FIG. 6A to FIG. 6C are diagrams for explaining processing in an extracting unit according to the first embodiment;

FIG. 7A to FIG. 7C are diagrams for explaining processing in a joining unit according to the first embodiment;

FIG. 8 is a diagram for explaining processing in a display control unit according to the first embodiment;

FIG. 9 is a flowchart for explaining processing in an ultrasound diagnosis apparatus according to a second embodiment;

FIG. 10 is a diagram for explaining processing in a joining unit according to the second embodiment;

FIG. 11 is a block diagram illustrating an exemplary configuration of an ultrasound diagnosis apparatus according to another embodiment; and

FIG. 12 is a block diagram illustrating an exemplary configuration of an image processing apparatus according to still another embodiment.

### DETAILED DESCRIPTION

An ultrasound diagnosis apparatus according to an embodiment includes an image generating unit, an extracting unit, and a joining unit. The image generating unit generates first volume data based on a result of transmission and reception of ultrasound waves that are executed when an ultrasound probe is located at a first position of a subject. The image generating unit also generates second volume data based on a result of transmission and reception of ultrasound waves that are executed when the ultrasound probe is located at a second position different from the first position. Under a first constraint on the orientation of a section, the extracting unit extracts, from the first volume data, first sectional image data containing a structural object inside the subject and taken along a direction in which the structural object extends. Under a second constraint on the orientation of a section, the extracting unit also extracts, from the second volume data, second sectional image data containing the structural object and taken along the direction in which the structural object extends. The joining unit generates joined image data composed of at least a part of the first sectional image data and at least a part of the second sectional image data joined together.

The following describes ultrasound diagnosis apparatuses according to embodiments with reference to the drawings.

### First Embodiment

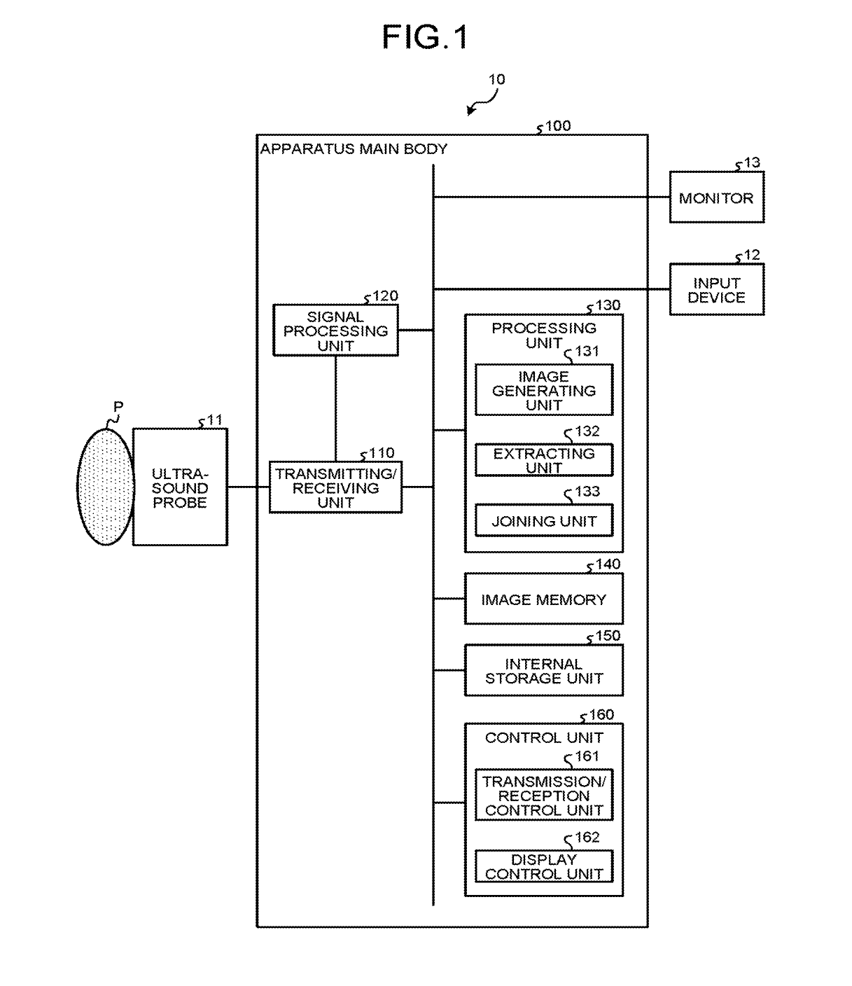

FIG. 1 is a block diagram illustrating an exemplary configuration of an ultrasound diagnosis apparatus 10 according to a first embodiment. As illustrated in FIG. 1, the ultrasound diagnosis apparatus 10 according to the first embodiment includes an ultrasound probe 11, an input device 12, a monitor 13, and an apparatus main body 100.

The ultrasound probe 11 is brought into contact with a body surface of a subject P and transmits and receives ultrasound waves. For example, the ultrasound probe 11 includes a plurality of piezoelectric transducer elements. These piezoelectric transducer elements generate ultrasound waves based on drive signals supplied from a transmitting/receiving unit 110 included in the apparatus main body 100 to be described later. The ultrasound waves generated are reflected in body tissue in the subject P and are received by the piezoelectric transducer elements in the form of reflected wave signals. The ultrasound probe 11 transmits the reflected wave signals received by the piezoelectric transducer elements to the transmitting/receiving unit 110.

The ultrasound probe 11 according to the first embodiment executes transmission and reception of ultrasound waves (scanning) on a three-dimensional region at a certain volume rate (frame rate). For example, the ultrasound probe 11 is a 2D array probe having a plurality of piezoelectric transducer elements arranged two-dimensionally in a grid-like pattern. The ultrasound probe 11 transmits ultrasound waves to a three-dimensional region through a plurality of piezoelectric transducer element arranged two-dimensionally and receives reflected wave signals. The ultrasound probe 11 is not limited to this example and may be, for example, a mechanical 4D probe that scans a three-dimensional region by causing a plurality of one-dimensionally arrayed piezoelectric transducer elements to mechanically swing.

The input device 12 includes a mouse, a keyboard, a button, a panel switch, a touch command screen, a foot switch, a track ball, a joystick, or the like, and receives various setting requests from an operator of the ultrasound diagnosis apparatus 10 and forwards the received various setting requests to the apparatus main body 100. The input device 12 is an example of an input unit.

The monitor 13 displays a graphical user interface (GUI) that the operator of the ultrasound diagnosis apparatus 10 uses for inputting various setting requests using the input device 12 and displays, for example, ultrasound image data generated in the apparatus main body 100.

The apparatus main body 100 is an apparatus that generates ultrasound image data based on the reflected wave signals received by the ultrasound probe 11. As illustrated in FIG. 1, the apparatus main body 100 includes, for example, the transmitting/receiving unit 110, a signal processing unit 120, a processing unit 130, an image memory 140, an internal storage unit 150, and a control unit 160. The transmitting/receiving unit 110, the signal processing unit 120, the processing unit 130, the image memory 140, the internal storage unit 150, and the control unit 160 are communicably connected to one another.

The transmitting/receiving unit 110 controls transmission and reception of ultrasound waves that are executed by the ultrasound probe 11. For example, based on instructions from the control unit 160 to be described later, the transmitting/receiving unit 110 controls transmission and reception of ultrasound waves that are executed by the ultrasound probe 11. The transmitting/receiving unit 110 applies drive signals (drive pulses) to the ultrasound probe 11, thereby causing an ultrasound beam to be transmitted into which ultrasound waves are focused in a beam shape. The transmitting/receiving unit 110 performs addition processing by assigning certain delay times to reflected wave signals received by the ultrasound probe 11, thereby generating reflected wave data in which reflection components are emphasized from a direction agreeing with the reception directivity of the reflected wave signals.

The signal processing unit 120 applies various kinds of signal processing to the reflected wave data generated from the reflected wave signals by the transmitting/receiving unit 110. The signal processing unit 120 applies, for example, logarithmic amplification and envelope detection processing to the reflected wave data received from the transmitting/receiving unit 110, thereby generating data (B-mode data) in which the signal intensity of each sample point (observation point) is expressed in brightness of luminance.

The signal processing unit 120 also generates, from the reflected wave data received from the transmitting/receiving unit 110, data (Doppler data) into which pieces of motion information of a moving body based on the Doppler effect are extracted at sample points in a scanning region. Specifically, the signal processing unit 120 generates Doppler data into which average speeds, dispersion values, power values or the like are extracted as the pieces of motion information of the moving body at the respective sample points. Here, examples of the moving body include a blood flow, tissue of a cardiac wall, and a contrast agent.

The processing unit 130 performs, for example, processing for generation of image data (ultrasound image data) and various kinds of image processing on image data. The processing unit 130 stores, in the image memory 140, image data generated and image data subjected to various kinds of image processing. The processing unit 130 is an example of a processing circuitry.

The processing unit 130 according to the first embodiment includes an image generating unit 131, an extracting unit 132, and a joining unit 133. The image generating unit 131 generates ultrasound image data from data generated by the signal processing unit 120. For example, from B-mode data generated by the signal processing unit 120, the image generating unit 131 generates B-mode image data in which the intensity of a reflected wave is expressed in luminance. The image generating unit 131 also generates Doppler image data representing moving body information from the Doppler data generated by the signal processing unit 120. The Doppler image data is speed image data, dispersion image data, power image data, or image data obtained by combining any of the foregoing data. When volume data is to be displayed, the image generating unit 131 generates two-dimensional image data for display by performing various kinds of rendering processing on the volume data. Processing that the extracting unit 132 and the joining unit 133 perform is to described later.

The image memory 140 is a memory that stores therein image data generated by the image processing unit 131. The image memory 140 can also store therein data generated by the signal processing unit 120. The B-mode data and Doppler data stored in the image memory 140 can be called up, for example, by the operator after diagnosis, and are turned into ultrasound image data for display through the image generating unit 131.

The internal storage unit 150 stores therein: control programs for use in transmission and reception of ultrasound waves, image processing, and display processing; diagnosis information (such as patient IDs and doctor's opinions, for example); and various kinds of data such as diagnosis protocols and various body marks. The internal storage unit 150 is used also for, for example, archiving image data stored in the image memory 140, as need arises. Data stored in the internal storage unit 150 can be transferred to an external device via an interface unit (not illustrated).

The control unit 160 controls all processing in the ultrasound diagnosis apparatus 10. Specifically, based on various setting requests input from the operator via the input device 12 and various control program and various data loaded from the internal storage unit 150, the control unit 160 controls processing in units such as the transmitting/receiving unit 110, the signal processing unit 120, and the processing unit 130. The control unit 160 causes the monitor 13 to display ultrasound image data stored in the image memory 140. The control unit an example of a processing circuitry.

The control unit 160 according to the first embodiment include a transmission/reception control unit 161 and a display control unit 162. Processing that the transmission/reception control unit 161 and the display control unit 162 perform is to be described later.

Each of the units such as the transmitting/receiving unit 110 or the control unit 10 that are embedded in the apparatus main body 100 may be constructed with hardware such as a processor (a central processing unit (CPU), a micro-processing unit (MPU), or an integrated circuit) or alternatively constructed with a computer program configured as software-based modules.

Here, in generating image data that covers a range wider than the scanning region of the ultrasound probe 11, it sometimes happens that the operator (a doctor) may lose track of a structural object as an imaging target. For example, when being unfamiliar with such a manipulation, the operator would lose track of a structural object (such as a blood vessel) as an imaging target during the course of moving the ultrasound probe 11 little by little on the body surface of the subject P. In this case, the operator cannot continue subsequent imaging, and starts over again the above manipulation in order to generate image data that covers the wider range.

Given this situation, the ultrasound diagnosis apparatus 10 according to the present embodiment includes the following components to generate image data (hereinafter also referred to as “joined image data” or “panoramic image data”) that covers a wide range with a simple operation. That is, in the ultrasound diagnosis apparatus 10, the ultrasound probe 11 executes transmission and reception of ultrasound waves to and from a three-dimensional region at a certain volume rate. Each time volume data, namely, image data of a three-dimensional region, is acquired from transmission and reception of ultrasound waves, the extracting unit 132 extracts, from the volume data, a section containing the long axis of a structural object inside the body of a subject. Each time image data of a section is extracted, a joining unit 133 generates image data composed of the extracted image data of a section and previously extracted image data of a section arranged at their respective corresponding positions. The display control unit 162 displays an image based on image data.

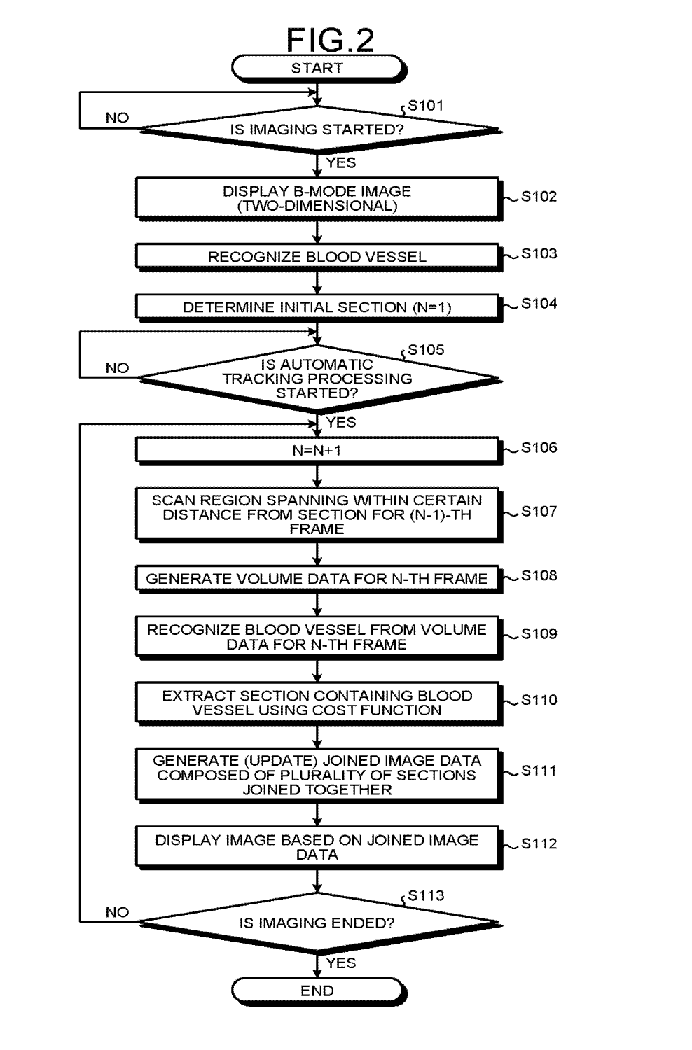

Processing in the above-described extracting unit 132, joining unit 133, transmission/reception control unit 161, and display control unit 162 is individually described by use of a flowchart in FIG. 2. Although the following descriptions refer a case where a blood vessel in a leg part (thigh) of the subject P is imaged as an imaging target that spans a range wider than the scanning region of the ultrasound probe 11, the embodiment is not limited to this case. The imaging target may be, for example, any structural object such as an esophagus that spans a range wider than the scanning region of the ultrasound probe 11. For example, the structural object is a tubular structural object such as a blood vessel or an esophagus.

FIG. 2 is a flowchart for explaining processing in the ultrasound diagnosis apparatus 10 according to the first embodiment. In imaging according to the first embodiment, an initial section with a blood vessel visualized therein determined first, and processing (automatic tracking processing) for enlarging images while tracking the blood vessel is then performed.

As illustrated in FIG. 2, if imaging is started (Yes at Step S101), the ultrasound diagnosis apparatus 10 performs processing for determining the initial section. For example, an operator brings the ultrasound probe 11 into contact with the leg part of the subject and presses a button for indicating the start of imaging. This acts as a trigger for the ultrasound diagnosis apparatus 10 to start the processing for determining the initial section. If imaging is not started (No at Step S101), the ultrasound diagnosis apparatus 10 beeps on standby.

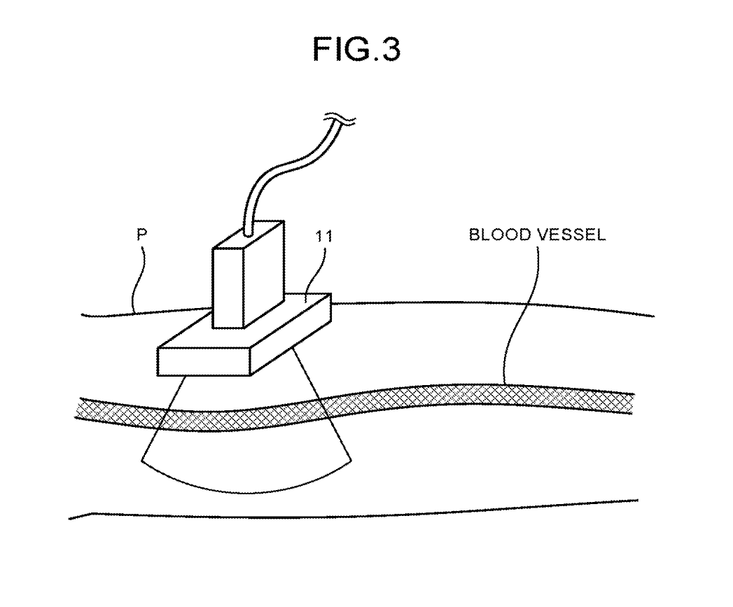

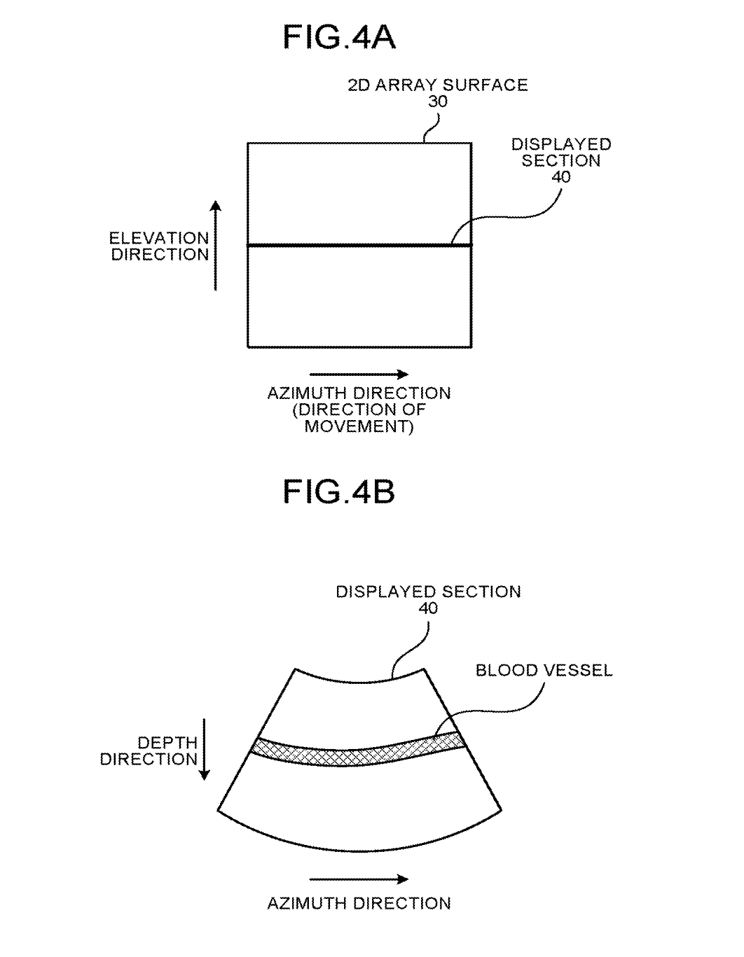

FIG. 3, FIG. 4A, and FIG. 48 are diagrams for explaining determination of the initial section according to the first embodiment. FIG. 3 illustrates how the ultrasound probe 11 is brought into contact with the subject P. FIG. 4A illustrates the position of a displayed section that is displayed in determination of the initial section. FIG. 4B illustrates the displayed section that is displayed in determination of the initial section.

As illustrated in FIG. 3, for example, the ultrasound probe 11, which is a 2D array probe, is brought into contact with the leg part of the subject. The ultrasound probe 11 then scans a certain section in order to determine the initial section. Here, the 2D array probe is also capable of scanning a two-dimensional (planer) region, for example, by causing piezoelectric transducer elements in one line to transmit and receive ultrasound waves.

Here, the ultrasound probe 11 has, as illustrated in FIG. 4A, a 2D array surface 30 on which a plurality of piezoelectric transducer elements are two-dimensionally arrayed in an azimuth direction and in an elevation direction. Here, the ultrasound probe 11 is moved by the operator in the azimuth direction. In this case, the ultrasound probe 11 scans, at a position at the center in the elevation direction, a section (the displayed section 40) paralleling the azimuth direction. Consequently, the ultrasound diagnosis apparatus 10 generates and displays a B-mode image of this displayed section 40, as illustrated in FIG. 4B, (Step 102).

Although the following descriptions continue with the case where the ultrasound probe 11 is moved in the azimuth direction, the embodiment is not limited to this case. For example, when the ultrasound probe 11 is moved in the elevation direction, the ultrasound probe 11 scans a section that paralleling the elevation direction.

Subsequently, in the ultrasound diagnosis apparatus 10, the extracting unit 132 recognizes the blood vessel (Step S103). For example, the extracting unit 13 recognizes the blood vessel using luminance values in a B-mode image. It has been known that a blood vessel appears as a black void against tissue (a solid part) surrounding the blood vessel. Therefore, the extracting unit 132 recognizes a blood vessel by extracting, from the B-mode image, a part appearing as a black void against tissue (a solid part) surrounding the part. The display control unit 162 then highlights, on the B-mode image, the position of the blood vessel recognized by the extracting unit 132 (refer to FIG. 4B). Processing for recognizing a blood vessel from a E-mode image is not limited to the above processing. For example, the transmission/reception control unit 161 may run both B-mode scanning and Doppler-mode scanning and recognize, as a blood vessel, a region having Doppler information (for example, a region the power value of which is greater than or equal to a threshold) in a Doppler image thus generated. Alternatively, a blood vessel may be specified manually by the operator.

Here, the operator moves the position of the ultrasound probe 11 while viewing a B-mode image from which a blood vessel has been recognized, thereby searching for a position that allows the blood vessel (imaging target) to be clearly visualized in the B-mode image. Subsequently, upon determining that the blood vessel has been clearly visualized in the B-mode image, the operator immobilizes the ultrasound probe 11 at the position and presses a button for determining an initial section. Consequently, the transmission/reception control unit 161 determines, as the initial section, a displayed section 40 that is being displayed when the button for determining an initial section is pressed (Step S104). That is, the input device 12 receives designation of a sectional position for extracting sectional image data. The transmission/reception control unit 161 then sets, as the first (N=1) frame, the displayed section 40 being currently displayed. Determination of the initial section is completed through the above-described part of processing.

Returning to description of FIG. 2, automatic tracking processing is described. After the determination of the initial section, if the operator presses a button for starting the automatic tracking processing, the individual processing units in the control unit 160 start the automatic tracking processing (Yes at Step S105). If the button for starting the automatic tracking processing is not pressed, the automatic tracking processing is not started (No at Step S105). In this case, for example, the initial section may be redetermined (corrected) by executing the processing Steps S102 to S104 again.

If the automatic tracking processing is of started (Yes at Step S105), the transmission/reception control unit 161 increments N by 1 (Step S106). The transmission/reception control unit 161 then scans a region within a certain distance from a section for a previous frame (the (N−1)-th frame) (Step S107).

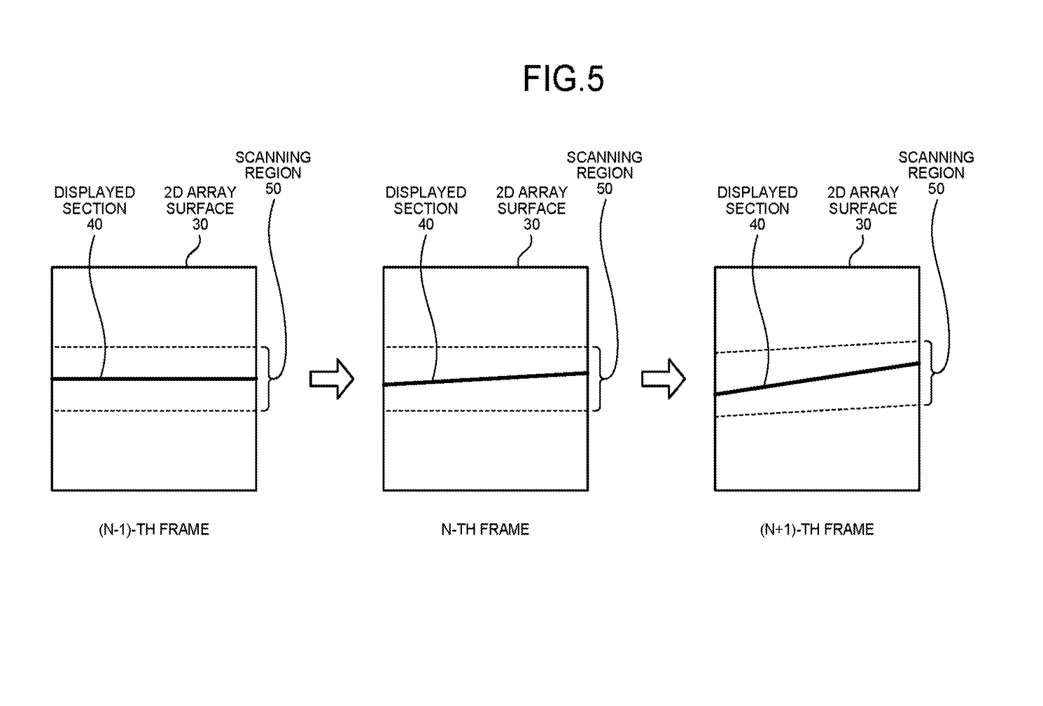

FIG. 5 is a diagram for explaining processing in the transmission/reception control unit 161 according to the embodiment. FIG. 5 illustrates a scanning region (search range) 50 that scanned in each frame by the ultrasound probe 11. As illustrated in FIG. 5, for example, the transmission/reception control unit 161 determines the scanning region 50 for the N-th frame, based on the position of a section for the (N−1)-th frame.

In one example, a description is given of a case where the scanning region 50 for the second (N=2) frame is determined. That is, the displayed section 40 for the (N−1)-th frame in FIG. 5 corresponds to the initial section (N=1). In this case, the transmission/reception control unit 161 sets, as the scanning region 50, a region (region inside the dash lines) that is a certain distance away in the elevation direction from the displayed section 40 set as the initial section. The transmission/reception control unit 161 then causes the ultrasound probe 11 to scan this scanning region 50 that is set based on the initial section. Subsequent sectional image data is extracted from this scanning region 50. That is, a sectional position for extracting sectional image data for the N-th frame depends on a sectional position for extracting sectional image data for the N-th frame.

That is, in scanning for the second frame, the transmission/reception control unit 161 causes scanning to be executed on a scanning region 50 that parallels a displayed section 40 (the initial section) for the first frame. Subsequently, in scanning for the third frame, the transmission/reception control unit 161 causes scanning to be executed on a scanning region 50 that parallels a displayed section 40 for the second frame.

The transmission/reception control unit 161 thus causes the ultrasound probe 11 to transmit and receive ultrasound waves to and from a scanning region 50 in a three-dimensional region and within the certain distance away from a section extracted from within previous volume data.

Returning to FIG. 2, further descriptions are made. After the ultrasound probe 11 executes scanning for the N-th frame, the image generating unit 131 generates volume data, based on three-dimensional reflected wave data in the N-th frame (Step S108). For example, each time volume data is generated, the image generating unit 131 stores the generated volume data in the image memory 140. That is, based on results of transmission and reception of ultrasound waves that are sequentially executed by the ultrasound probe 11, the image generating unit 131 generates time-series volume data.

Here, the operator carries out scanning while moving the ultrasound probe 11 little by little on the body surface of the subject P. That is, after scanning for the (N−1)-th frame is executed at a first position of the subject P, scanning for the N-th frame is executed at a second position different from the first position. That is, the image generating unit 131 generates first volume data based on a result of transmission and reception of ultrasound waves that are executed when an ultrasound probe 11 is located at the first position of the subject P. The image generating unit generates second volume data based on a result of transmission and reception of ultrasound waves that are executed when the ultrasound probe 11 is located at the second position. The first volume data and the second volume data are included in the time-series volume data.

The extracting unit 132 then recognizes a blood vessel from volume data in the N-th frame (Step S109). For example, each time volume data for the N-th frame is stored in the image memory 140, the extracting unit 132 recognizes a blood vessel from the volume data. In processing for recognizing a blood vessel, recognition may be carried out using luminance values (a black void) or may be carried out using Doppler information as described above. That is, the extracting unit 132 may recognize, as a blood vessel, a part in volume data that appears as a black void against tissue (a solid part) surrounding the part or may recognize, as a blood vessel, positions of sample points having Doppler information.

The extracting unit 132 then, by using a cost function, extracts image data (sectional image data) of a section that contains the blood vessel (Step S110). For example, the extracting unit 132 extracts image data of a section in which the extracted blood vessel is visualized in the longest length and the widest width.

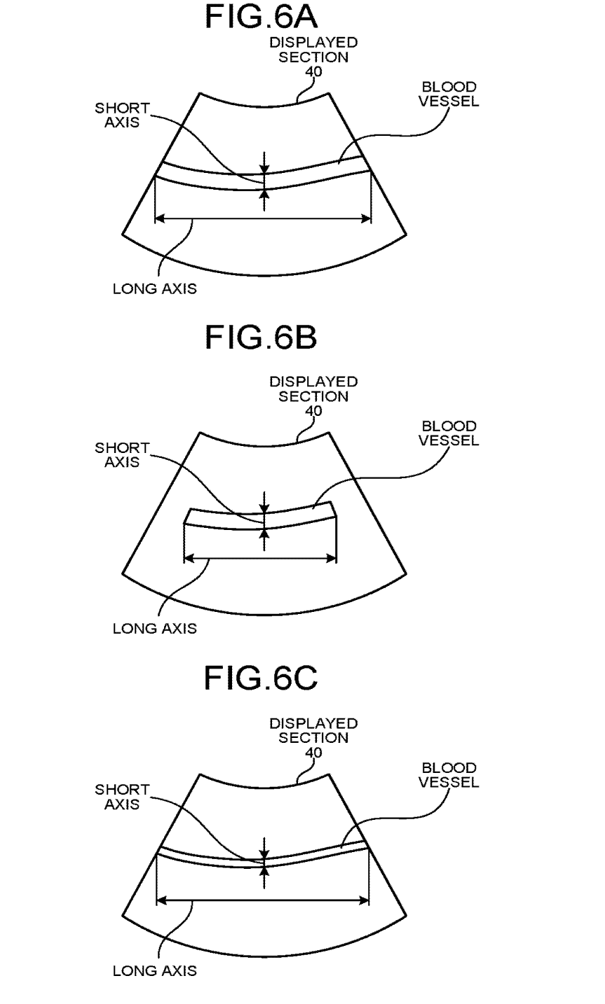

FIG. 6A to FIG. 6C, are diagrams for explaining processing in the extracting unit 132 according to the first embodiment. FIG. 6A to FIG. 6C illustrate displayed sections 40 for one frame each with a blood vessel visualized therein.

As illustrated in FIG. 6A to FIG. 6C, for example, from the volume data for the N-th frame, the extracting unit 132 generates a plurality of pieces of image data of sections that contain a blood vessel. Specifically, the extracting unit 132 generates a plurality of pieces of image data of sections that pass through a blood vessel recognized and parallel a depth direction (a direction in which the ultrasound probe 11 transmits and receives ultrasound waves). For example, the extracting unit 132 generates pieces of image data of displayed sections 40 that are illustrated in FIG. 6A to FIG. 6C, respectively.

The extracting unit 132 then, by using a cost unction given below as Mathematical Formula (1), extracts image data of a section that has the extracted blood vessel visualized in the longest length and the widest width, from among the generated pieces of image data. The cost function given as Mathematical Formula (1) is a function for evaluating the respective lengths of the long axis and the short axis of a blood vessel. While length_{short axis} denotes the length of the short axis, length_{long axis} denotes the length of the long axis. In addition, α and β are weighting coefficients.

Cost function=α×length_{short axis}+β×length_{long axis} (1)

That is, Mathematical Formula (1) is a function that evaluates the respective lengths of the long axis and the short axis of a structural object with certain weighting coefficients by plugging certain values in α and β, respectively. The respective values for α and β may be changed as desired. For example, the weighting coefficient α for the short axis direction may be set to 0, so that only an evaluation on the length in the long axis direction may be made. However, in consideration of convenience for generating joined image data, it is preferable that the value for the weighting coefficient β for the long axis direction be set to a value larger than 0.

For example, the extracting unit 132 acquires the lengths of the long axis and the short axis of the blood vessel from each of the respective pieces of the image data in FIG. 6A to FIG. 6C. For example, the extracting unit acquires the length of the long axis by assuming it to be the horizontal length in each section, and acquires the length of the short axis by assuming it to be the vertical length therein.

The extracting unit 132 then plugs the lengths thus acquired of the long axis and the short axis into Mathematical Formula (1) given above, thereby finding an evaluation value. Here, among FIG. 6A to FIG. 6C, the blood vessel in FIG. 6A is the widest and the largest. The blood vessel in FIG. 6B is shorter than the one in FIG. 6A. The blood vessel in FIG. 6C is narrower than the one in FIG. 6A. In such a case, the extracting unit 132 extracts the piece of image data in FIG. 6A as a piece of image data of a section having the blood vessel visualized in the longest length and the widest width.

Thus, each time volume data is acquired through transmission and reception of ultrasound waves, the extracting unit 132 extracts, from the volume data, image data of a section that contains the long axis of a structural object inside the body of the subject P. The reason that the extracting unit 132 performs processing using the long axis of a structural object is to extract sectional image data that extends along a direction in which the structural object extends. That is, the extracting unit 132 extracts, from the first volume data, first sectional image data containing a structural object inside the subject and taken along a direction in which the structural object extends, and also extracts, from the second volume data, second sectional image data containing the structural object and taken along a direction in which the structural object extends. Specifically, the extracting unit 132 extracts, as the second sectional image data, image data of a section containing the same site as a part of the structural object contained in the first sectional image data.

Although FIG. 6A to FIG. 60 illustrate, as an example, a case where image data of a section passing through a blood vessel and paralleling the depth direction is extracted, the embodiment is not limited to this case. For example, from within volume data, image data of a section may be extracted that passes through the center of a contact portion between the body surface and the ultrasound probe 11 and also through the center line of a blood vessel. Alternatively, image data of a section may be extracted that passes through the center line of a blood vessel and extends along the direction of gravitational force. The direction of gravitational force can be detected, for example, by having a position sensor attached to the ultrasound probe 11. In addition, for example, image data of an extracted section does not necessarily need to be planar. For example, the extracting unit 132 may extract image data of a curved surface that extends along directions in which a blood vessel (structural object) extends. Consequently, sectional image data following a curved surface continuing from the initial section can be sequentially extracted.

Although FIG. 6A to FIG. 6C illustrate, as one example, a case where a section is extracted from three sections, the embodiment is not limited to this case, and a section may be extracted from more than three sections, for example. However, it is preferable that sections from which a section is extracted be limited to those paralleling a certain direction (the depth direction in the foregoing example) so that the processing load can be kept down. Sections from which the section is extracted are not limited to those paralleling a certain direction, and may alternatively be, for example, sections allowed to incline to some extent from the certain direction and included in a certain range. Here, a section included in the certain range means, for example, a section included in a range obtained by rotating, a certain angle (for example, 3 degrees) with the axis of rotation positioned at the center line of a blood vessel, a section that passes through the center line of the blood vessel and parallels a certain direction. That is, the extracting unit 132 may extract a section included in a certain angular range of rotation the axis of which is positioned at the center line of a blood vessel. In other words, the extracting unit 132 may extract image data of sections under the constraint that the sections be included in a certain angular range of rotation the axis of which is positioned at the center line of a structural object.

The above certain angular range may be, for example, set on the basis of a section extracted in a frame immediately prior to the current one. For example, when extracting image data of a section for the N-th frame, the extracting unit 132 may extract a section included in a range that a section for the (N−1)-th frame passes when rotated a certain angle (for example, in units of 3 degrees) with the axis of rotation positioned at the center line of a blood vessel. In other words, the extracting unit 132 may extract image data of a section for the N-th frame under the constraint that the section be included in a certain angular range of rotation the axis of which is positioned at the center line of a structural object. The certain angular range is set on the basis of a section for the (N−1)-th frame.

The extracting unit 132 thus extracts image data of a section from volume data for each frame under a constraint on the orientation of the section. That is, under a first constraint on the orientation of a section, the extracting unit 132 extracts, from the first volume data, first sectional image data that contains a structural object inside the subject and that is taken along a direction in which the structural object extends. Under a second constraint or the orientation of a section, the extracting unit 132 also extracts, from the second volume data, second sectional image data that contains the structural object and that is taken along the direction in which the structural object extends.

For example, the extracting unit 132 extracts the first sectional image data under a first constraint that sectional image data according to the orientation of the ultrasound probe 11 when the ultrasound probe 11 is located at a first position be extracted. In one example, the extracting unit 132 extracts the first sectional image data under a constraint that the sectional image data be contained in a direction paralleling the orientation of the ultrasound probe 11 (that is, the depth direction) or in a certain angular range of rotation the axis of which is positioned at the center line of the structural object.

In addition, for example, the extracting unit 132 extracts the second sectional image data under a constraint that sectional image data according to the orientation of the first sectional image data be extracted. In one example, the extracting unit 132 extracts image data of a section for the N-th frame under a constraint that the section be contained in a certain angular range of rotation the axis of which positioned at the center line of the structural object, the angular range being set on the basis of section for the (N−1) -th frame.

The specific contents of the fir constraint and the second constraint described above are the same as each other. However, the specific contents of the first constraint and the second constraint do not necessarily need to be the same as each other. For example, the angular range of rotation in the second constraint may be 2 degrees while the angular range of rotation in the first constraint is 3 degrees. In addition, for example, the processing for acquiring the lengths of the long axis and the short axis of a structural object is not limited to the above example. For example, the extracting unit 132 may acquire the lengths by assuming, within a plurality of pixels forming a blood vessel, a line segment obtained by connecting the two most distant pixels as the long axis and a line segment perpendicular to the long axis as the short axis.

Returning to FIG. 2, further descriptions are made. If image data of a section is extracted by the extracting unit 132, the joining unit 133 generates (updates) joined image data composed of pieces of image data of a plurality of sections joined together (Step S111). For example, each time image data of the displayed section 40 for the N-th frame is extracted, the joining unit 133 generates the joined image data by joining together the image data of the displayed section 40 for the N-th frame and the image data of the displayed section 40 for the (N−1)-th frame. Consequently, the joining unit 133 updates joined image data 70 already generated up to the (N−1)-th frame.

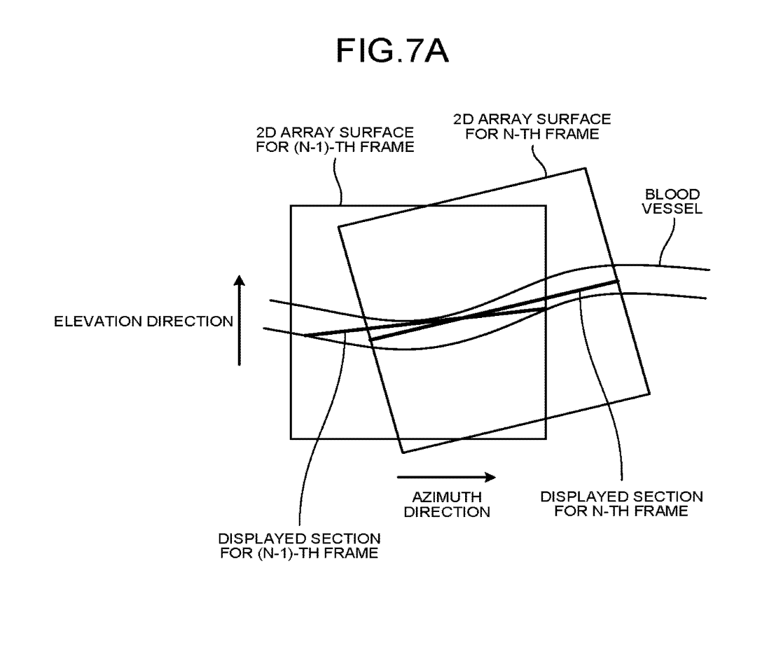

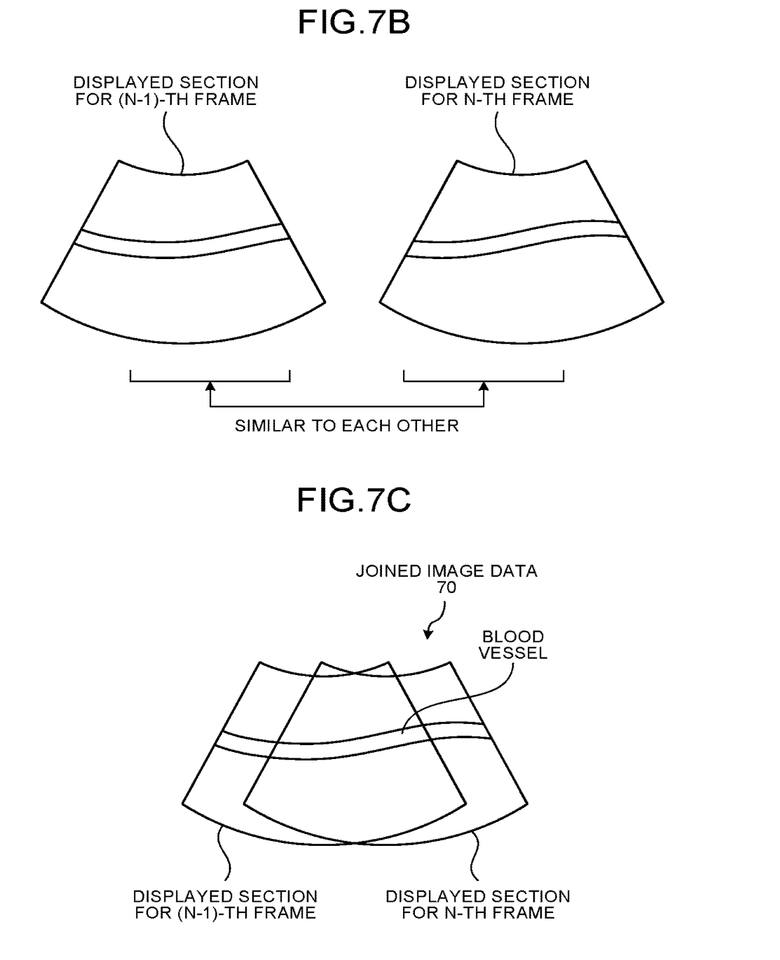

FIG. 7A to FIG. 7C are diagrams for explaining processing in the joining unit 133 according to the first embodiment. FIG. 7A illustrates the positional relation between the displayed sections 40 for the N-th frame and the (N−1)-th frame. FIG. 7B illustrates respective pieces of image data of the displayed sections 40 for the N-th frame and the (N−1)-th frame. FIG. 7C illustrates the joined image data 70 generated by the joining unit 133.

As illustrated in FIG. 7A, scanning is executed with the ultrasound probe 11 (that is, the 2D array surface 30) moved along the body surface. Therefore, the displayed sections 40 for the N-th frame and the (N−1)-th frame are located near to each other. In the example in FIG. 7A, along the azimuth direction, the right-hand side of the displayed section 40 for the (N−1)-th frame and the left-hand side of th displayed section 40 for the N-th frame are located near to each other. For this reason, as illustrated in FIG. 7B, the right-hand side of the displayed section 40 for the (N−1)-th frame and the left-hand side of the displayed section 44 for the N-th frame are similar to each other. Given this situation, as illustrated in FIG. 7C, the joining unit 133 generates the joined image data 70 by overlapping these similar ranges on each other. Exemplary manners of “joining” here include: cutting out two pieces of image data at the same positions in terms of orientation and direction and combine pieces of image data obtained by the cutting out into one; and combining a range of image data of one of the two pieces of image data and the other piece of image data, the range being other than the similar range. When the two pieces of image data are on the same plane in a three-dimensional space, these pieces may be combined into one by obtaining pixel values of the overlapping ranges by a statistical method (such as averaging, finding the maximum, or finding the minimum).

Specifically, if image data of the section for the N-th frame is extracted, the joining unit 133 performs pattern matching (an image recognition technique) between the image data of the section for the N-th frame and image data of section for the (N−1)-th frame using characteristic points (such as edges or corners) of a structural object contained in both of the two pieces of image data, thereby matching the positions of the two pieces of image data with each other. Specifically, the joining unit 133 obtains the most similar positions by a similar image determination method using the sum of absolute differences (SAD), the sum of squared differences (SSD), the Normalized Cross-Correlation (NCC), or the like as an evaluation function. The joining unit 133 then joins together the two pieces of image data at corresponding positions (that is, the most similar positions) in the two pieces of image data. Here, the joining unit 133 performs alpha blending (weighted synthesis) to synthesize ranges that are similar to each other in the two pieces of image data. That is, the joining unit 133 joins together at least a part of first sectional image data and at least a part of second sectional image data so that a part of a structural object in the first sectional image data and a part of the structural object in the second sectional image data can continue into each other. Consequently, the joining unit 133 generates the joined image data 70 such that corresponding contours of the structural object in the two pieces of image data can continue into each other.

Each time image data of a section is extracted, the joining unit 133 generates the joined image data 70 composed of extracted image data of a section and previously extracted image data of a section arranged at their respective corresponding positions. For example, when image data of the displayed section 40 for the N-th frame is extracted, the joined image data 70 is updated by joining that image data of the displayed section 40 with the joined image data 70 already generated up to the (N−1)-th frame, Consequently, the joining unit 133 can generate image data that accurately reproduces the length of the structural object (blood vessel) inside the body of the subject in the azimuth direction. As illustrated in FIG. 7C, pieces of image data that are joined together do not necessarily need to be joined together in such a manner that the respective entireties thereof are jointed together. That is, the extracting unit 132 generates joined image data composed of at least a part of first sectional image data and at least part of second sectional image data joined together.

Processing in the joining unit 133 is not limited to the above descriptions. For example, the joining unit 133 does not necessarily need to perform weighted synthesis. For example, as illustrated in FIG. 7A, when the displayed sections 40 of two pieces of image data cross each other, one side of the line of intersection of the crossing may be generated from the displayed section 40 for the (N−1)-th frame and the other side thereof may be generates the displayed section 40 for the N-th frame.

Also for example, the joining unit 133 may perform pattern matching using a common region shared by respective pieces of volume data for the N-th frame and the (N−1)-th frame, to match the positions of the two pieces of volume data with each other. The joining unit 133 may then generate the joined image data 70 by, based on the result of this position matching, joining together image data of respective displayed sections 40 for the N-th frame and the (N−1)-th frame.

When a blood vessel is extremely winding, image data having a blood vessel visualized the length of which in the azimuth direction is short may be acquired without having the blood vessel visualized as having a sufficient length (for example, refer to FIG. 6B). In this case, the joining unit 133 does not necessarily need to use the entire region of image data of the displayed section 40. In generation of the joined image data 70, the joining unit 133 may use, for example, image data obtained by removing the right and left parts of the displayed section 40 so that the image can be made shorter in the azimuth direction to fit with the length of the visualized blood vessel.

Returning to FIG. 2, further descriptions are made. If the joining unit 133 generates (updates) the joined image data 70, the display control unit 162 displays an image based on the joined image data 70 (Step S112). For example, each time the joining unit 133 updates the joined image data 70, the display control unit 162 displays the updated joined image data 70 on the monitor 13.

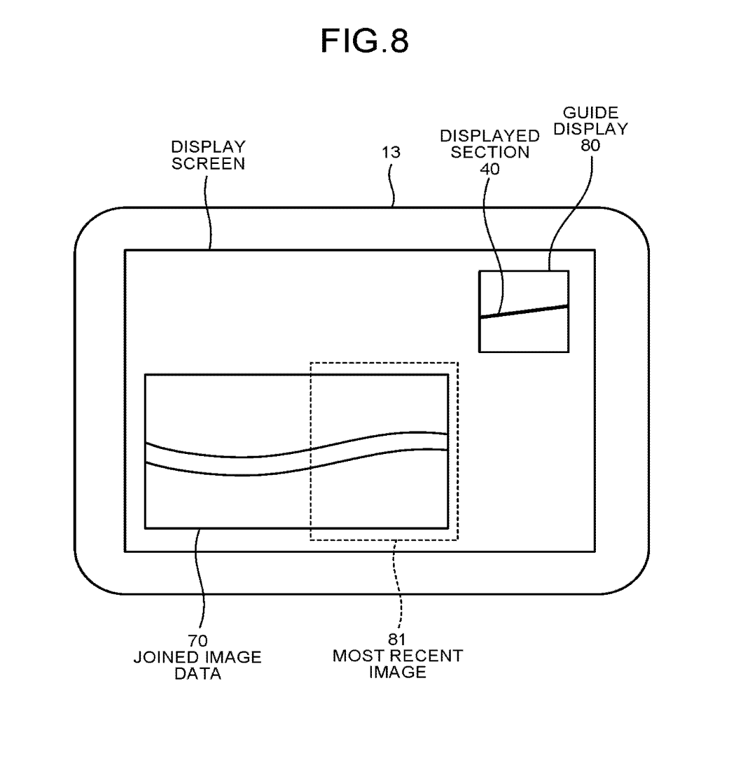

FIG. 8 is a diagram for explaining processing in the display control unit 162 according to the first embodiment. FIG. 8 illustrates one example of a display screen displayed on the monitor 13 by the display control unit 162. Specifically, on the display screen of the monitor 13 illustrated in FIG. 8, an image based on the joined image data 70 and a guide display 80 for indicating the position of a blood vessel as an imaging target are displayed.

As illustrated in FIG. 8, the display control unit 162 generates, based on the joined image data 70, an image to be displayed and displays the image on the monitor 13. For example, when the rightward direction in FIG. 8 corresponds to the direction of movement of the ultrasound probe 11, a most recent image 81 is located at the rightmost end of the joined image data 70. In this case, the display control unit 162 generates, from image data contained in the joined image data 70 and within a certain distance (length) from the rightmost end thereof, an image to be displayed and displays the image. Consequently, regardless of how long the joined image data 70 is extended, the display control unit 162 can display, on a certain reduced scale, a joined image containing the most recent image 81.

For example, the display control unit 162 also displays the guide display 80 on the display screen of the monitor 13. This guide display 80 corresponds to image data indicating the position of a displayed section 40 in a three-dimensional region that can be imaged by the ultrasound probe 11. For example, when the extracting unit 132 extracts image data of a displayed section 40, the display control unit 162 acquires, from the extracting unit 132, information indicating the position of the displayed section 40 relative to the 2D array surface 30. Subsequently, based on the information acquired from the extracting unit 132, the display control unit 162 generates, as the guide display 80, image data indicating the position of the most recent displayed section 40 (a displayed section 40 for the N-th frame) relative to the 2D array surface 30 and displays the image. The display control unit 162 then displays the guide display 80. That is, the position of the displayed section 40 in the guide display 80 corresponds to the position of the most recent image 81. Consequently, the display control unit 162 can display the position of the most recent displayed section 40 in a three-dimensional region that can be imaged by the ultrasound probe 11. In other words, by moving the ultrasound probe 11 while viewing the guide display 80, the operator can reduce the risk of losing track of a structural object as an imaging target.

The display control unit 162 thus displays an image based on the joined image data 70. Processing in the display control unit 162 is not limited to the above descriptions. For example, the display control unit 162 may display the entire region of the generated joined image data 70 on the monitor 13. Also for example, when a displayed section 40 is likely to deviate from the 2D array surface 30, the display control unit 162 may notify the operator thereof. For example, when the length of the displayed section 40 in the guide display 80 is shorter than a certain threshold (length), the display control unit 162 displays a message saying “you may be losing track of a blood vessel”, causes the guide display 80 to flash, or changes the color of the guide display 80. The display control unit 162 may highlight the most recent image 81 so that the operator can be aware of where it is.

As described above, the ultrasound diagnosis apparatus 10 repeats executing the processing at Step S106 to Step S112 so long as the imaging is not ended (No at Step S113), thereby extending the joined image data 70. Subsequently, if the imaging is ended (Yes at Step S113), the ultrasound diagnosis apparatus 10 ends the automatic tracking processing and ends the processing for extending the joined image data 70.

A processing procedure in the ultrasound diagnosis apparatus 10 is not limited to the processing procedure illustrated in FIG. 2. For example, although Step S104 for determining the initial section and Step S105 for starting the automatic tracking processing are executed as different steps of processing in the case described using FIG. 2, the embodiment is not limited to this case. For example, Step S104 and Step S105 may be executed as the same step of processing. In this case, for example, if an operation that determines the initial section is performed, this operation acts as a trigger to start the automatic tracking processing.

As described above, in the ultrasound diagnosis apparatus 10 according to the first embodiment, the ultrasound probe 11 executes transmission and reception of ultrasound waves to and from a three-dimensional region at a certain volume rate. Each time volume data, namely, image data of a three-dimensional region, is acquired from transmission and reception of ultrasound waves, the extracting unit extracts, from the volume data, a section containing the long axis of a structural object inside the body of a subject. Each time image data of a section is extracted, the joining unit 133 generates image data having the extracted image data of a section and previously extracted image data of a section arranged at their respective corresponding positions. The display control unit 162 displays an image based on the image data. Therefore, the ultrasound diagnosis apparatus 10 enables image data that covers a wide range to be generated with a simple operation.

For example, as long as the structural object as an imaging target is contained in a scanning region being scanned by the ultrasound probe 11, the ultrasound diagnosis apparatus 10 automatically extracts, from volume data thereof, image data of a section visualizing the long axis of the structural object, and generates (updates) the joined image data 70. Therefore, by moving the ultrasound probe 11 so that the structural object can be contained in a three-dimensional scanning region, the operator can easily generate the joined image data 70 having the structural object visualized therein. That is, without, manually positioning a scanned section with respect to the structural object, the operator can easily generate the joined image data 70 having the structural object visualized therein.

For example, in the ultrasound diagnosis apparatus 10, the transmission/reception control unit 161 causes the ultrasound probe 11 to transmit and receive ultrasound waves to and from the scanning region 50 located, in a three-dimensional region, within the certain distance from a section extracted from previous volume data. By thus being configured, the transmission/reception control unit 161 does not run scanning on all over a region that can be scanned by the ultrasound probe 11 (that is, the entire region of the 2D array surface 30) but runs scanning on a limited region. The frame rate (volume rate) can be thus improved. This additionally results in a smaller size of volume data for each frame, and therefore, for example, a processing load on the extracting unit 132 that performs processing on volume data can be reduced. Specifically, the extracting unit 132 can have a reduced number of sections to be generated from volume data, and therefore can have a reduced processing load thereon. In addition, the extracting unit 132 can have a reduced number of sections, and therefore can accurately extract a section that has the structural object visualized more suitably.

The above embodiment describes a case where a plurality of pieces of volume data including first and second volume data are generated by sequentially (for example, at certain time intervals) performing volume scanning while moving the ultrasound probe 11. However, the embodiment is not limited to this case. For example, the embodiment may alternatively be implemented in such a manner that the volume scanning is performed with a button pressed that is provided on the apparatus main body 100 or the ultrasound probe 11 for requesting scanning. In this case, the operator, for example, generates the first volume data by pressing the button while putting the ultrasound probe 11 in contact with a certain position on the subject, and then generates the second volume data by pressing the button after changing the position to another. A plurality of pieces of volume data are generated by repeating the operation of thus pressing the button each time the position of the ultrasound probe 11 is changed.

The embodiment is not limited to the button for requesting scanning and may alternatively be implemented, for example, in such a manner that, with the movement of the ultrasound probe 11 detected, volume scanning is executed at the timing when the ultrasound probe 11 stops. In this case, for example, the operator generates first volume data by stopping, at desired timing (position), movement of the ultrasound probe 11 being moved along the body surface of the subject. Then, after restarting movement of the ultrasound probe 11, the operator generates the second volume data by stopping the movement again at desired timing. A plurality of pieces of volume data are generated by repeating such operation that stops the movement of the ultrasound probe 11 at desired timing.

When movement, of the ultrasound probe 11 is restarted before the completion of volume scanning, volume data being generated by this volume scanning remains incomplete. In this case, for example, the incomplete volume data may be discarded without being used in the above processing (extraction and joining of sectional image data). That is, in case of incomplete volume data, volume data generated immediately before the incompletion is used in the above processing.

The above embodiment describes the case where the scanning region of the volume data for the N-th frame is narrowed down based on the position of a section for the (N−1)-th frame so that a search range from which image data of a section is extracted can be narrowed down refer to FIG. 5). However, the embodiment is not limited to this case. For example, the scanning region does not necessarily need to he narrowed down as long as the search range has already been narrowed down. That is, the extracting unit 132 may determine a search range through the same processing as processing for determining the scanning region 50, which is illustrated in FIG. 5, and extract image data of a section from the determined search range. In this case, for example, the transmission/reception control unit 161 may cause the ultrasound probe 11 to scan, for all of the frames, all over regions that can be scanned thereby (that is, the entire region of the 2D array surface 30).

### Second Embodiment

In the first embodiment, the case where image data for each frame is generated and joined along a direction (depth direction) in which ultrasound waves are transmitted and received is described. The embodiment is not limited to this case. For example, the ultrasound diagnosis apparatus 10 may join together respective pieces of volume data for frames and displays any desired section.

An ultrasound diagnosis apparatus 10 according to a second embodiment includes the same constituent elements as the ultrasound diagnosis apparatus 10 illustrated in FIG. 1, and differs therefrom in parts of processing that the joining unit 133 and the display control unit 162 perform. For this reason, the points different from the first embodiment are mainly described in the second embodiment, and descriptions of the points having the same functions as those described in the first embodiment are omitted.

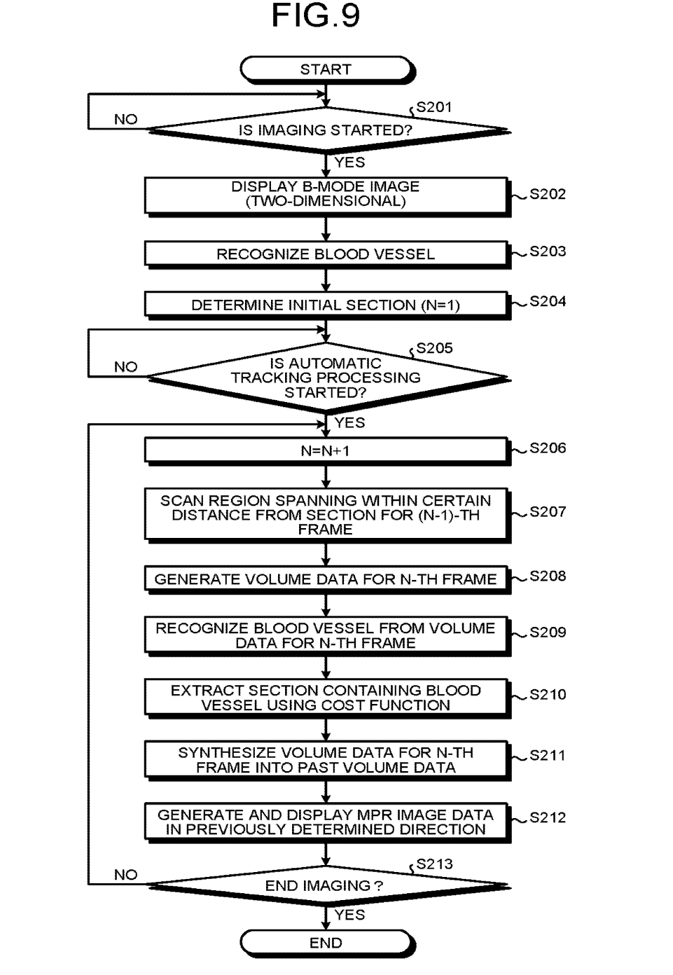

Through a flowchart in FIG. 9, processing in the ultrasound diagnosis apparatus 10 according to the second embodiment is explained. FIG. 9 is a flowchart for explaining processing in the ultrasound diagnosis apparatus 10 according to the second embodiment. Respective steps of processing in Step S201 to Step S210 illustrated in FIG. 9 are the same as the respective steps of processing in Step S101 to Step S110 illustrated in FIG. 2, and descriptions thereof are therefore omitted.

As illustrated in FIG. 9, after the extracting unit 132 extracts a section, the joining unit 133 synthesizes volume data for the N-th frame with past volume data (Step S211). For example, each time a displayed section 40 for the N-th frame is extracted, the joining unit 133 matches the position of the volume data for the N-th frame with the position of volume data for the (N−1)-th frame, thereby generating joined volume data composed of these two pieces of volume data joined together.

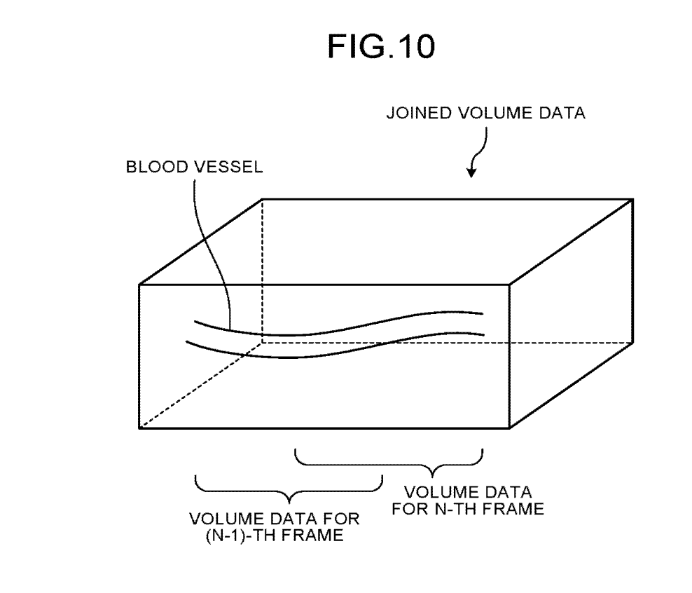

FIG. 10 is a diagram for explaining processing in the joining unit 133 according to the second embodiment. FIG. 10 illustrates an example of the joined volume data composed of the volume data for the N-th frame and the volume data for the (N−1)-th frame joined together.

Here, as illustrated in FIG. 7A, scanning is executed with the ultrasound probe 11 (that is, the 2D array surface 30) moved along the body surface. Therefore, respective scanning regions for the N-th frame and the (N−1)-th frame share a common region. For this reason, the respective pieces of volume data for the N-th frame and the (N−1)-th frame share a common region.

Given this situation, as illustrated in FIG. 10, the joining unit 133 performs pattern matching using a common region shared by the respective pieces of volume data for the N-th frame and the (N−1)-th frame to position these two pieces of volume data with each other. The joining unit 133 joins together the two pieces of volume data by superimposing corresponding positions therein on each other. Here, the joining unit 133 performs alpha blending to synthesize regions in the two pieces of volume data that are a common region shared thereby. Consequently, the joining unit 133 generates joined volume data.

The joining unit 133 thus synthesizes the volume data for the N-th frame with past volume data, thereby generating (updating) the joined volume data. That is, as the ultrasound probe 11 is moved, the joined volume data (and a blood vessel) illustrated in FIG. 10 is (are) updated in a direction of movement thereof. Also in joining volume data, as described in the first embodiment, any of the following is applicable: cutting out two pieces of volume data and combining the cut-out pieces thereof into one; and combining a range of volume data of one of the two pieces of volume data and the other piece of volume data into one, the range being other than a similar range. Alternatively, the two pieces of volume data may be combined into one by obtaining pixel values of the overlapping ranges by a statistical method.

Returning to FIG. 9, further descriptions are made. After generating the joined volume data, the joining unit 133 performs multi planar reconstruction (MPR) processing on the joined volume data to generate MPR image data in a previously designated direction, and the display control unit 162 displays the MPR image data (Step S212). For example, the extracting unit 132 generates the MPR image data under the constraint that the MPR image data pass through the center line of a blood vessel and parallel the direction of gravitational force.

As an example, a case where an operator previously designates a section to be displayed that contains the long axis of a blood vessel recognized in all frames and that parallels the 2D array surface 30. In this case, each time the joined volume data is updated, the joining unit 133 executes MPR processing on the updated joined volume data to generate MPR image data that cuts the blood vessel along a section paralleling the 2D array surface 30. The display control unit 162 then displays the NPR image data generated by the joining unit 133 on a display screen of the monitor 13.

The ultrasound diagnosis apparatus 10 repeats executing the processing at Step S206 to Step S212 so long as the imaging is not ended (No at Step S213), thus generating (updating) the joined volume data. Subsequently, if the imaging is ended (Yes at Step S213), the ultrasound diagnosis apparatus 10 ends the automatic tracking processing and ends the processing for generating the joined volume data.

In the ultrasound diagnosis apparatus 10 according to the second embodiment, the joining unit 133 generates joined volume data composed of first volume data and second volume data joined together. Under a constraint on the orientation of a section, the extracting unit 132 then extracts, from the joined volume data, sectional image data containing the structural object inside the body of the subject and taken along the direction in which the structural object extends. This configuration enables the ultrasound diagnosis apparatus 10 to, for example, provide sections of a blood vessel of a subject along various directions. This configuration therefore enables the operator to observe the state of a blood vessel from various directions, thereby making the ultrasound diagnosis apparatus 10 useful in, for example, diagnoses of arteriosclerosis obliterans and aneurysm. For example, the operator is enabled to observe a plaque site, even though it is unobservable in a certain section, in another section.

A sectional position that is extracted in the above MPR processing is not limited to being previously determined and, for example, may be designated by the operator at the timing when an MPR section is displayed. In this case, for example, the input device 12 receives designation of a first sectional position that is used for extracting the first sectional image data. Specifically, the input device 12 receives an operation that designates, as the position of an MPR section, an angle of rotation about the center line of a blood vessel. In this case, for example, the display control unit 162 displays, as a GUI to be used for inputting an angle of rotation, an image of a section perpendicular to the center line of the blood vessel. In this image, the center line of the blood vessel is visualized as the center point of the image, and the position of the MPR section is visualized as a straight line passing though the center line. This straight line is rotatable about the position of the center line (the center point). That is, the operator can designate an angle of the MPR section about the center line by rotating (changing the angle of) this straight line to any desired angle. In other words, upon receiving, from the operator, an operation that designates an angle of rotation the axis of which is positioned at the center line of a structural object, the extracting unit 132 extracts, from the joined volume data, sectional image data located at the angle of rotation designated by the operation.

The specific details described in the first embodiment are also applicable to the second embodiment other than to generating joined volume data and generating an MPR image data from the generated joined volume data.

### Other Embodiments

Embodiments according to the present disclosure can be implemented in various different forms other than the foregoing embodiments.

### Automatic Setting of Initial Section

For example, although the cases where the initial section is determined when it is designated (a button is pressed) by an operator are described in the above embodiments, embodiments are not limited to these cases. For example, the cost function given as Mathematical Formula (1) may be used also in determination of the initial section to automatically determine the initial section.

### Use of Position Sensor

For example, although the cases where generating the joined image data 70 (or the joined volume data) involves performing the position matching through pattern matching are described in the above embodiments, embodiments are not limited to these cases. For example, positional information from a position sensor may be used for this position matching.

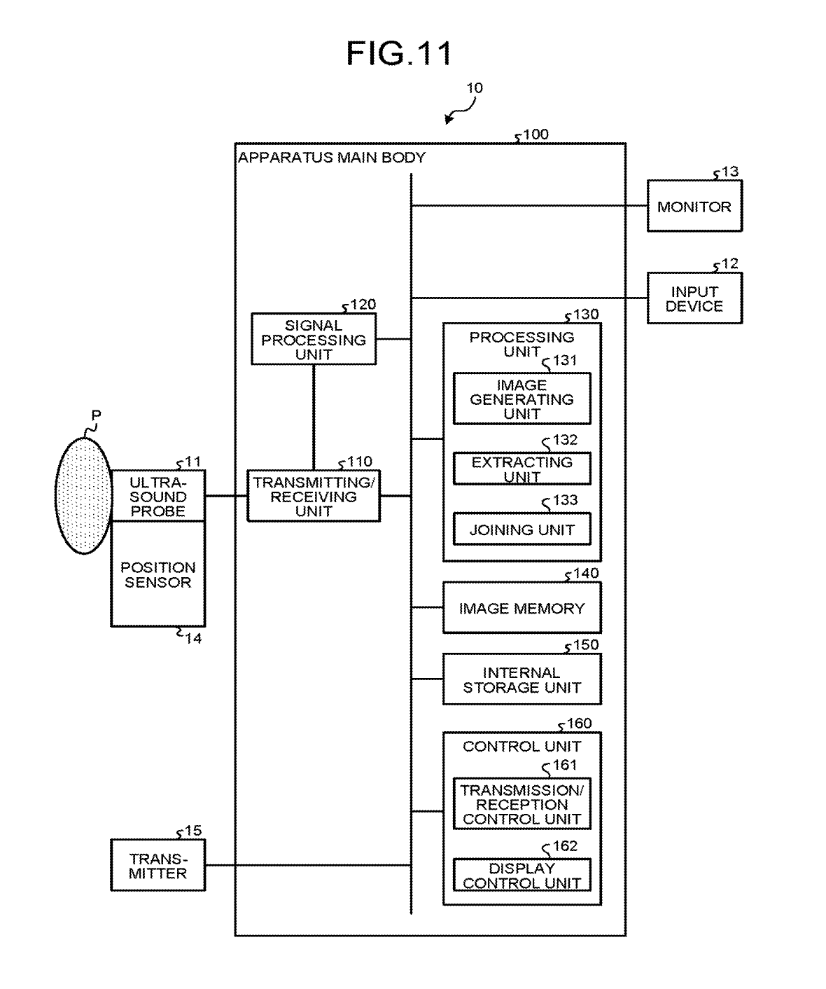

FIG. 11 is a block diagram illustrating an exemplary configuration of an ultrasound diagnosis apparatus 10 according to another embodiment. As illustrated in FIG. 11, an ultrasound diagnosis apparatus 10 according to this other embodiment includes the same constituent elements as the ultrasound diagnosis apparatus 10 illustrated in FIG. 1, and differs therefrom in further including a position sensor 14 and a transmitter 15 and in parts of processing that the joining unit 133 performs.

The position sensor 14 and the transmitter 15 are devices for acquiring positional information on the ultrasound probe 11. For example, the position sensor 14 is a magnetic sensor that is attached to the ultrasound probe 11. Also for example, the transmitter 15 is a device that is arranged at any desired position and forms a magnetic field oriented outward with the transmitter 15 at its center.

The position sensor 14 detects a three-dimensional magnetic field formed by the transmitter 15. Subsequently, based on information on the detected magnetic field, the position sensor 14 calculates the position (coordinates and angle) of itself in a space in which the origin is located at the transmitter 15, and transmits the calculated position to the control unit 160. Here, the position sensor 14 transmits positional information on itself, that is, positional information on the ultrasound probe 11, in individual frames to the control unit 160. Consequently, the joining unit 133 can acquire the positional information in the individual frames from the position sensor 14.

The joining unit 133 matches the positions of image data of sections in the individual frames with one another by using the positional information in the respective frames that has been acquired from the position sensor 14. For example, once a section for the N-th frame is extracted, the joining unit 133 matches the positions of image data of the section for the N-th frame and of image data of a section for the (N−1)-th frame with each other using the positional information in the N-th frame and the positional information in the (N−1)-th frame. The joining unit 133 then performs matching between these two pieces of image data with positions that have been matched with each other using the positional information at the center. The joining unit 133 can thus more accurately match the positions of the two pieces of image data with each other. The joining unit 133 then joins together the two pieces of image data at corresponding positions (that is, the most similar positions) in the two pieces of image data.

The joining unit 133 thus matches the positions of the individual frames with one another by using the positional information in the respective frames that has been acquired from the position sensor 14. Consequently, the joining unit 133 can increase the processing speed while improving the accuracy of the position matching. The joining unit 133 can similarly use the positional information in matching the positions of volume data with each other.

Although a case of acquiring positional information on the ultrasound probe 11 using a magnetic sensor is instanced in the example illustrated in FIG. 11, embodiments are not limited to this case. For example, positional information on the ultrasound probe 11 may be acquired using any one device selected from a three-dimensional acceleration sensor, a three-dimensional gyro sensor, and a three-dimensional compass instead of a magnetic sensor or may be acquired using an appropriate combination of any two or more of the above devices.

### Contrast Agent

For example, the cases where no contrast agent is used are described in the above embodiments, embodiments are not limited to these cases. For example, the use of a contrast agent in the above processing enables the ultrasound diagnosis apparatus 10 to generate the joined image data 70 while additionally detecting a blood vessel that cannot be detected without a contrast agent.

### Image Processing Apparatus

The processing described in each of the foregoing embodiments may be executed in an image processing apparatus.

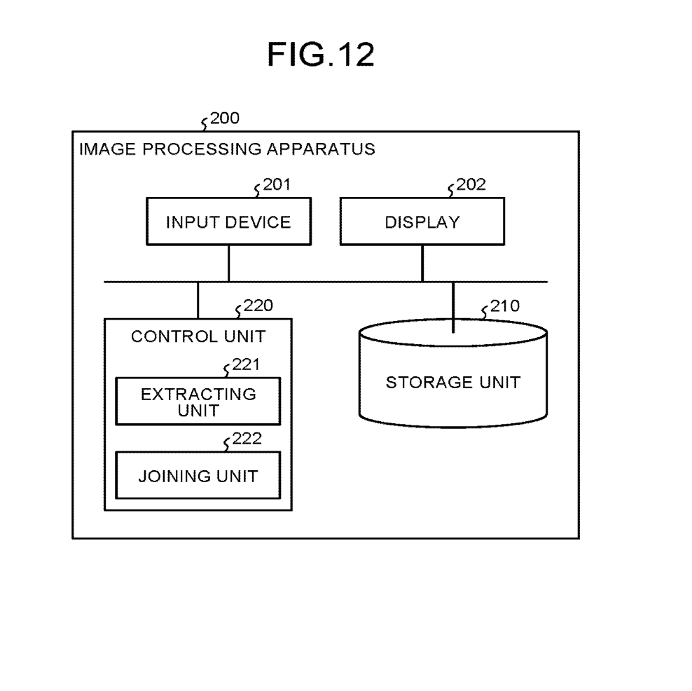

FIG. 12 is a block diagram illustrating an exemplary configuration of an image processing apparatus according to still another embodiment. As illustrated in FIG. 12, an image processing apparatus 200 includes an input device 201, a display 202, a storage unit 210, and a control unit 220.

The input device 201 includes a mouse, a keyboard, a button, a panel switch, a touch command screen, a foot switch, a track ball, a joystick, or the like, and receives various setting requests from an operator of the image processing apparatus 200 and forwards the received various setting requests to individual processing units.

The display 202 displays a GUI that the operator of the image processing apparatus 200 uses for inputting various setting requests using the input, device 201 and displays, for example, information generated in the image processing apparatus 200.

The storage unit 210 is a non-volatile storage device, the examples of which include a semiconductor memory device such as flash memory, a hard disk, and an optical disc.

The storage unit 210 stores therein a volume data similar to the volume data generated by the image generating unit 131 described in the first and second embodiments. That is, the storage unit 210 stores first volume data generated based on a result of transmission and reception of ultrasound waves that are executed when the ultrasound probe 11 is located at a first position of a subject. The storage unit 210 also generates second volume data generated based on a result of transmission and reception of ultrasound waves that are executed when the ultrasound probe is located at a second position different from the first position.

The control unit 220 is an integrated circuit such as an application specific integrated circuit (ASIC) or a field programmable gate array (FPGA) or an electronic circuit such as a CPU or an MPU, and controls all processing in the image processing apparatus 200.

The control unit 220 includes an extracting unit 221 and a joining unit 222. The extracting unit 221 and the joining unit 222 have functions similar to those of the extracting unit 132 and the joining unit 133 described in the first and second embodiments, respectively. That is, the extracting unit 221 extracts, from the first volume data, first sectional image data containing a structural object inside the subject and taken along a direction in which the structural object extends, and also extracts, from the second volume data, second sectional image data containing the structural object and taken along a direction in which the structural object extends. The joining unit 222 generates joined image data composed of at least a part of the first sectional image data and at least part of the second sectional image data joined together. Specific details of processing in the extracting unit 221 and the joining unit 222 are the same as those in the foregoing embodiments, and descriptions thereof are therefore omitted. By being thus configured, the image processing apparatus 200 enables image data that covers a wide range to be generated with a simple operation.

The various constituent elements of the various devices and apparatuses illustrated in the explanation of the above-described embodiments are functionally conceptual, and do not necessarily need to be configured physically as illustrated. That is, the specific forms of distribution or integration of the devices and apparatuses are not limited to those illustrated, and the whole or a part thereof can be configured by being functionally or physically distributed or integrated in any form of units, depending on various types of loads, usage conditions, and the like. Furthermore, the whole of or a part of the various processing functions that are performed in the respective devices and apparatuses can be implemented by a CPU and a computer program to be executed by the CPU, or can be implemented as hardware by wired logic.

For example, although the cases where the ultrasound diagnosis apparatus 10 separately includes the processing unit 130 and the control unit 160, embodiments are not limited to those cases. For example, the ultrasound diagnosis apparatus 10 may have the functions of the processing unit 130 and functions of the control unit 160 incorporated into a single processing circuit. Of the respective steps of processing described in the above embodiments, the whole or a part of those described as being configured to be automatically performed can be manually performed, or the whole of a part of those described as being configured to be manually performed can be automatically performed by known methods. In addition, the processing procedures, the control procedures, the specific names, and the information including various data and parameters including various kinds of data and parameters described herein and illustrated in the drawings can be optionally changed unless otherwise specified.

The image processing method described in the foregoing embodiments can be implemented by executing a previously prepared image processing program on a computer such as a personal computer or a workstation. This image processing program can be distributed via a network such as the Internet. The image processing program can also be recorded on a computer-readable recording medium such as a hard disk, a flexible disk (FD), a compact disc read only memory (CD-ROM), a magnetic optical disc (MO), or a digital versatile disc (DVD), and executed by being read out from the recording medium by the computer.

According to at least one of the embodiments described above, image data that covers a wide range can be generated with a simple operation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising processing circuitry configured to:
generate first volume data based on a result of transmission and reception of ultrasound waves that are executed when an ultrasound probe is located at a first position of a subject, and generate second volume data based on a result of transmission and reception of ultrasound waves that are executed when the ultrasound probe is located at a second position different from the first position;
extract first sectional image data from the first volume data under a first constraint on an orientation of a section, and extract second sectional image data from the second volume data under a second constraint on an orientation of a section, the first sectional image data containing a structural object inside the subject and being taken along a direction in which the structural object extends, the second sectional image data containing the structural object and being taken along a direction in which the structural object extends; and
generate joined image data composed of at least a part of the first sectional image data and at least a part of the second sectional image data joined together.

2. The ultrasound diagnosis apparatus according to claim 1, wherein the processing circuitry extracts the first sectional image data under a constraint that sectional image data according to an orientation of the ultrasound probe when the ultrasound probe is located at the first position be extracted.

3. The ultrasound diagnosis apparatus according to claim 1, wherein the processing circuitry extracts the second sectional image data under a constraint that sectional image data according to an orientation of the first sectional image data be extracted.

4. The ultrasound diagnosis apparatus according to claim 1, wherein the specific contents of the first constraint and the second constraint are the same.

5. The ultrasound diagnosis apparatus according to claim 1, wherein the processing circuitry extracts, as the second sectional image data, image data of a section containing the same site as a part of the structural object contained in the first sectional image data.

6. The ultrasound diagnosis apparatus according to claim 1, wherein the processing circuitry joins together at least a part of first sectional image data and at least a part of second sectional image data in such a manner that a part of the structural object in the first sectional image data and a part of the structural object in the second sectional image data continue into each other.

7. The ultrasound diagnosis apparatus according to claim 1, wherein
based on results of transmission and reception of ultrasound waves that are sequentially executed by the ultrasound probe, the processing circuitry generates time-series volume data, and
the first volume data and the second volume data are included in the time-series volume data.

8. The ultrasound diagnosis apparatus according to claim 1, wherein
the processing circuitry receives designation of a first sectional position that is used for extracting the first sectional image data, and
a second sectional position that is used for extracting the second sectional image data depends on the first sectional position.

9. The ultrasound diagnosis apparatus according to claim 8, wherein
the structural object is a tubular structural object, and
the designation of the first sectional position is an operation to designate an angle of rotation about the center line of the tubular structural object contained in the first volume data.

10. The ultrasound diagnosis apparatus according to claim 1, further comprising:
transmission/reception control circuitry configured to cause the ultrasound probe to execute transmission and reception of ultrasound waves to and from a region defined as being within a certain distance from the first sectional image data, wherein
the ultrasound probe executes transmission and reception of ultrasound waves to and from the region, and
based on a result of transmission and reception of ultrasound waves that have been executed to and from the region, the processing circuitry generates the second volume data.

11. The ultrasound diagnosis apparatus according to claim 1, wherein the processing circuitry extracts at least one of the first sectional image data and the second sectional image data by using a function for evaluating a length of the structural object along a direction in which the structural object extends.

12. The ultrasound diagnosis apparatus according to claim 1, wherein the processing circuitry causes an image based on the joined image data to be displayed, and further causes sectional-position image data to be displayed relative to a region that is scannable by the ultrasound probe, the sectional-position image data indicating a position of a section corresponding to the most recent piece of sectional image data joined together in the joined image data.

13. An ultrasound diagnosis apparatus comprising processing circuitry configured to:
generate first volume data based on a result of transmission and reception of ultrasound waves that are executed when an ultrasound probe is located at a first position of a subject, and generate second volume data based on a result of transmission and reception of ultrasound waves that are executed when the ultrasound probe is located at a second position different from the first position;
generate joined volume data composed of the first volume data and the second volume data joined together; and
under a constraint on an orientation of a section, extract, from the joined volume data, sectional image data containing a structural object inside a body of the subject and taken along a direction in which the structural object extends.

14. The ultrasound diagnosis apparatus according to claim 13, wherein, upon receiving, from an operator, an operation that designates an angle of rotation the axis of which is positioned at the center line of the structural object, the processing circuitry extracts, from the joined volume data, sectional image data corresponding to the angle of rotation designated by the operation.
